(19) United States
(12) Patent Application Publication
(10) Pub. No.: US 2015/0150935 A1
(43) Pub. Date: Jun. 4, 2015

(54) PERIPHERAL KAPPA RECEPTOR AGONISTS FOR REDUCING PAIN AND INFLAMMATION

(71) Applicant: CARA THERAPEUTICS, INC., Sheldon, CT (US)

(72) Inventors: Derek T. Chalmers, Riverside, CT (US); James B. Jones, Metuchen, NJ (US); Robert H. Spencer, New Hope, PA (US)

(73) Assignee: CARA THERAPEUTICS, INC., Shelton, CT (US)

(21) Appl. No.: 14/403,535

(22) PCT Filed: Jun. 5, 2013
(86) PCT No.: PCT/US2013/044302

§ 371 Date: Nov. 24, 2014

Related U.S. Application Data
(60) Provisional application No. 61/655,731, filed on Jun. 5, 2012.

(51) Int. Cl. A61K 38/07; A61K 31/485; A61K 9/00; A61K 31/40

(52) U.S. Cl. CPC A61K 38/07; A61K 31/40; A61K 31/485; A61K 9/0019

(57) ABSTRACT

A method of treating of a mammalian subject suffering from an inflammatory disease or condition by administering a peripherally-restricted kappa opioid receptor agonist for reducing the inflammation is provided. The peripherally-restricted kappa opioid receptor agonist can include a peptide and the peptide can include D-amino acids. Administration of peripherally-restricted kappa opioid receptor agonists results in lowering of serum levels of pro-inflammatory cytokines and elevation of levels of anti-inflammatory cytokines.

### RELATED APPLICATION

The present application claims the benefit of U.S. provisional patent application Ser. No. 61/655,731 filed Jun. 5, 2012, the disclosure of which is incorporated by reference herein in its entirety.

### FIELD OF THE INVENTION

The present invention relates to the prevention, inhibition or treatment of inflammation, especially inflammation resulting from surgical procedures or other bodily insult. More particularly, the invention relates to the administration of peripherally-restricted kappa opioid receptor agonists to reduce post-surgical inflammation.

### RELATED ART

Non-narcotic analgesics, such as the non-steroidal anti-inflammatory drugs (NSAIDs) have been used for the management and treatment of pain and inflammation. However, the NSAIDs (e.g. ibuprofen) also have unwanted side effects such as hepatotoxicity and ulcers and gastric bleeding due to Cox1 activity. There is a need for analgesics and anti-inflammatory agents that are free of these side effects of NSAIDs.

Morphine acts at the mu opioid receptor to produce its analgesic effects. Compounds acting at the kappa opioid receptor can relieve pain and also suppress inflammation. U.S. Pat. No. 5,965,701 to Junien et al. discloses kappa receptor agonists which have an affinity for the KOR at least 1,000 times their affinity for the mu opioid receptor and an ED₅₀ of not greater than about 0.5 mg/kg. U.S. Pat. Nos. 7,402,564; 7,727,963 and 7,713,937 disclose synthetic peptide amides that are kappa opioid receptor ligands and exhibit low P₄₅₀ CYP inhibition and low penetration into the brain. U.S. Pat. No. 7,842,662 discloses dimeric forms of the synthetic peptide amides that retain their kappa receptor agonist properties.

U.S. patent application Ser. No. 12/768,686 discloses the administration of the kappa opioid agonist synthetic peptide amides by intravenous, oral and topical routes. US patent application publication no. 2010/0075910 discloses the use of the kappa opioid agonist synthetic peptide amides for the treatment of inflammatory pain.

The morphine-sparing effects of one such the kappa opioid agonist synthetic peptide amide, CR845, administered to women after laparoscopic-assisted hysterectomy has been reported (Menzaghi et al., 2010 13^{th} World Congress on Pain). In this reported Phase 2 clinical trial, subjects were administered a 15-minute intravenous infusion of 0.04 mg/kg CR845 or matching placebo following recovery from surgery and reaching a pain intensity baseline level of from 5 to 8 on a scale of zero to 11. Pain intensity was assessed up to 8 hours after the CR845 infusion or until morphine was administered by patient controlled analgesia. Morphine use was reduced by 32% over the first 16 hours post-infusion in CR845-treated patients versus placebo accompanied by a significant reduction in opioid side effects (vomiting, nausea and pruritis, i.e. itching) with no significant changes in clinical lab test results, vital signs, electrocardiograms, or Ramsey sedation scale assessment. CR845 was shown to be safe and well tolerated, and effective in reducing pain intensity when administered after surgery.

The link between cytokines and inflammation is well documented. See for instance: “Cytokines in Disease” Whicher, J. T. and S. W. Evans (1990) Clin. Chem. 36(7) 1269-1281; “Cytokines and Inflammation” Ed. E. S. Kimball, (1991) CRC Press, Inc., 2000 Boca Raton, Fla.; and “Cytokines and Inflammation” Aarons, A. and L. Borish, (1993) Immuno Methods 3(1) 3-12; and “Role of pro-and anti-inflammatory cytokines during inflammation: experimental and clinical findings” C. A. Dinarello J. Biological Regulators and Homeostatic Agents. (1997) 11: (3) 91-103.

### BRIEF DESCRIPTIONS OF THE FIGURES

FIG. 1 shows the design of the clinical trial described in Example 1. N: Number of patients; VAS: visual analog pain scale; PACU: post anesthesia care unit; Opioid Rescue prn: Pro re nata—Opioid rescue “as needed.”

FIG. 2 shows the relative levels of reduction of post-operative pain over the first 24 hours after surgery as demonstrated by the summed pain intensity difference (SPID) over all time points. Columns are mean values, and the bars are standard errors about the mean (SEM).

FIG. 3 shows the relative levels of reduction of post-operative pain over the first 24 hours after surgery as demonstrated by the pain intensity difference (PID) from zero to 24 hours.

FIG. 4 shows the amounts of morphine (in milligrams) self administered by the patients in each group: placebo-placebo, placebo-CR845, CR845-placebo, and CR845-CR845 in the intervals: 2-4 hours, 4-12 hours and 12-24 hours post surgery.

FIG. 5 shows the evaluations of pain relief as assessed by the patients themselves, demonstrating that pre-treatment with CR845 was consistently evaluated as very good or excellent by the highest percentage of patients.

FIG. 6 shows the results of C-reactive protein assays in samples of patient serum taken pre surgery and at 1 hour, 8 hours and 24 hours post laparoscopic hysterectomy after receiving two placebos or two doses of CR845.

FIG. 7 shows the results of IL-6 assays in samples of patient serum taken pre surgery and at 1 hour, 8 hours and 24 hours post laparoscopic hysterectomy after receiving two placebos or two doses of CR845.

FIG. 8A shows the results of TNFα assays and FIG. 8B shows the results of IL-1β assays in samples of patient serum taken at 1 hour, 8 hours and 24 hours post laparoscopic hysterectomy after receiving two placebos or two doses of CR845.

FIG. 9A shows the results of IL-2 assays and FIG. 9B shows the results of IL-8 assays in samples of patient serum taken at 1 hour, 8 hours and 24 hours post laparoscopic hysterectomy after receiving two placebos or two doses of CR845. The difference in the change from baseline level of IL-8 was highly significant (p=0.007).

FIG. 10A shows the results of IL-12 assays and FIG. 10B shows the results of GM-CSF assays in samples of patient serum taken at 1 hour, 8 hours and 24 hours post laparoscopic hysterectomy after receiving two placebos or two doses of CR845.

FIG. 11A shows the results of IL-6 assays and FIG. 11B shows the results of IL-7 assays in samples of patient serum taken at 1 hour, 8 hours and 24 hours post laparoscopic hysterectomy after receiving two placebos or two doses of CR845.

FIG. 12A shows the results of IL-4 assays and FIG. 12B shows the results of IFNγ assays from samples of patient serum taken at 1 hour, 8 hours and 24 hours post laparoscopic hysterectomy after receiving placebo or a single dose of CR845.

FIG. 13A shows the results of IL-5 assays and FIG. 13B shows the results of IL-10 assays in samples of patient serum taken at 1 hour, 8 hours and 24 hours post laparoscopic hysterectomy after receiving two placebos or two doses of CR845.

FIG. 14 shows the results of IL-13 assays from samples of patient serum taken at 1 hour, 8 hours and 24 hours post laparoscopic hysterectomy after receiving placebo or a single dose of CR845.

FIG. 15 shows the incidence of treatment emergent adverse events (TEAEs) including nausea, vomiting, pruritus and generalized pruritus through 24 hours after first infusion of CR845 after laparoscopic hysterectomy in a Phase IIb trial.

### SUMMARY

In one embodiment the present invention provides a method for reducing kappa opioid receptor-associated inflammation in a mammalian subject, wherein the method includes administering an effective amount of a peripherally-restricted kappa opioid receptor agonist to the subject. In one embodiment the inflammation is due to an inflammatory disease or condition. In another embodiment the inflammation is due to a medical procedure.

In another embodiment the present invention provides a method for reducing kappa opioid receptor-associated inflammation in a mammalian subject, the method includes administering an effective amount of a peripherally-restricted kappa opioid receptor agonist and wherein the administering of the peripherally restricted kappa opioid receptor agonist causes a reduction in the level of one or more pro-inflammatory cytokines and/or the increase in an level of one or more anti-inflammatory cytokines in the blood of the subject. In one embodiment the pro-inflammatory cytokines are chosen from IL-1β, IL-2, IL-4 IL-6, IL-7, IL-8, IL-12(p70), GM-CSF, TNFα and IFNγ. In another embodiment the anti-inflammatory cytokines are chosen from IL-5, IL-10 and IL-13.

The invention provides the first-in-man use of a non-narcotic kappa opioid receptor agonist analgesic used as a pretreatment prior to and/or during surgery to reduce pain and inflammation. The peripheral restriction of the kappa opioid receptor agonist analgesics of the invention provides the non-narcotic analgesic and anti-inflammatory properties due to action at peripheral kappa receptors and very limited penetration across the blood brain barrier, and therefore little or no action at the kappa receptors in the CNS and the brain.

In another embodiment, the present invention provides a method for reducing kappa opioid receptor-associated inflammation in a mammalian subject, the method comprising administering an effective amount of a peripherally restricted kappa opioid receptor agonist to the subject and thereby reducing the kappa opioid receptor-associated inflammation in the mammalian subject.

The kappa opioid receptor-associated inflammation can be any inflammatory disease or condition including, but not limited to sinusitis, rheumatoid arthritis tenosynovitis, bursitis, tendonitis, lateral epicondylitis, adhesive capsulitis, osteomyelitis, osteoarthritic inflammation, inflammatory bowel disease (IBD), irritable bowel syndrome (IBS), ocular inflammation, otitic inflammation or autoimmune inflammation.

In one embodiment, the present invention provides a method for reducing post medical procedure inflammation in a mammalian subject, the method comprising administering an effective amount of a peripherally restricted kappa opioid receptor agonist to the subject prior to the medical procedure that normally causes post-medical procedure inflammation and thereby reducing the post-medical procedure inflammation experienced by the subject.

The subject can be a human patient, a non-human primate or any other mammal. The non-human primate can be any non-human primate such as, for instance an ape, gorilla, orangutan, lemur, monkey or chimpanzee. The non-primate mammal can be the non-primate mammal such as a pet or companion animal, e.g. a dog or a cat; a high-value mammal such as a thoroughbred horse, a show animal or a farm animal, such as a cow, a goat, a sheep or a pig.

In another embodiment, the invention provides a method for reducing patient need for morphine, such as for instance as judged by patient controlled analgesia (PCA) demand.

In another embodiment, the peripherally restricted kappa opioid receptor agonist useful in the practice of the present invention includes a peptide. The peptide can include any amino acid, such as for instance a natural amino acid, an L-amino acid, a D-amino acid, or a non-natural amino acid, such as for instance, and without limitation, D-Ala(cyclopropyl, cyclobutyl, cyclopentyl or cyclohexyl), D-Ala (thienyl), D-Nle (i.e., D-norleucine) or (α-Me)D-Leu.

In another embodiment, the peptide includes at least four D-amino acids. In still another embodiment, the peripherally restricted kappa opioid receptor agonist includes a synthetic peptide amide having the formula I:

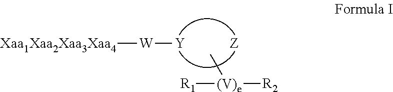

or a stereoisomer, mixture of stereoisomers, prodrug, pharmaceutically acceptable salt, hydrate, solvate, acid salt hydrate, N-oxide or isomorphic crystalline form thereof.

In one embodiment, the residue Xaa₁ can be any of the following: (A)(A′)D-Phe, (A)(A′)(α-Me)D-Phe, D-Tyr, D-Tic, D-tert-leucine, D-neopentylglycine, D-phenylglycine, D-homophenylalanine, and β-(E)D-Ala, and wherein each (A) and each (A′) are phenyl ring substituents independently selected from the group consisting of —H, —F, —Cl, —NO₂, —CH₃, —CF₃, —CN, and —CONH₂, and wherein each (E) is independently selected from the group consisting of cyclobutyl, cyclopentyl, cyclohexyl, pyridyl, thienyl and thiazolyl.

In another embodiment, the residue Xaa₂ is selected from the group consisting of (A)(A′)D-Phe, 3,4-dichloro-D-Phe, (A)(A′)(α-Me)D-Phe, D-1Nal, D-2Nal, D-Tyr, (E)D-Ala and D-Trp.

In another embodiment, the residue Xaa₃ is selected from the group consisting of D-Nle, D-Phe, (E)D-Ala, D-Leu, (α-Me)D-Leu, D-Hle, D-Val, and D-Met.

In another embodiment, the residue Xaa₄ is selected from the group consisting of (B)₂D-Arg, (B)₂D-Nar, (B)₂D-Har, ζ-(B)D-Hlys, D-Dap, ε-(B)D-Lys, ε-(B)₂-D-Lys, D-Amf, amidino-D-Amf, γ-(B)₂D-Dbu, δ-(B)₂α-(B′)D-Orn, D-2-amino-3(4-piperidyl)propionic acid, D-2-amino-3(2-aminopyrrolidyl)propionic acid, D-α-amino-β-amidinopropionic acid, α-amino-4-piperidine-acetic acid, cis-α,4-diaminocyclohexane acetic acid, trans-α,4-diaminocyclohexaneacetic acid, cis-α-amino-4-methylaminocyclo-hexane acetic acid, trans-α-amino-4-methylaminocyclohexane acetic acid, α-amino-1-amidino-4-piperidineacetic acid, cis-α-amino-4-guanidinocyclohexane acetic acid, and trans-α-amino-4-guanidinocyclohexane acetic acid, wherein each (B) is independently selected from —H and C₁-C₄ alkyl, and (B′) is H or (α-Me).

In another embodiment, the linking group, W is selected from the group consisting of: Null, provided that when W is null, Y is N; —NH—(CH₂)_{b}— with b equal to zero, 1, 2, 3, 4, 5, or 6; and —NH—(CH₂)_{c}—O— with c equal to 2, or 3, provided that Y is C.

In another embodiment, the moiety

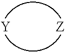

is an optionally substituted 4 to 8-membered heterocyclic ring moiety wherein all ring heteroatoms in said ring moiety are N; wherein Y and Z are each independently C or N.

In another embodiment, the moiety

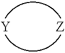

is an optionally substituted 4 to 8-membered heterocyclic ring moiety wherein all ring heteroatoms in said ring moiety are N; wherein Y and Z are each independently C or N, provided that when such ring moiety is a six, seven or eight-membered ring, Y and Z are separated by at least two ring atoms.

In another embodiment, the moiety

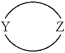

is an optionally substituted 4 to 8-membered heterocyclic ring moiety wherein all ring heteroatoms in said ring moiety are N; wherein Y and Z are each independently C or N provided that when such ring moiety has a single ring heteroatom which is N, then such ring moiety is non-aromatic; the group, V is C₁-C₆ alkyl, and e is zero or 1, wherein when e is zero, then V is null and R₁ and R₂ are directly bonded to the same or different ring atoms.

In another embodiment (i) R₁ is selected from the group consisting of —H, —OH, halo, —CF₃, —NH₂, —COOH, C₁-C₆ alkyl, C₁-C₆ alkoxy, amidino, C₁-C₆ alkyl-substituted amidino, aryl, optionally substituted heterocyclyl, Pro-amide, Pro, Gly, Ala, Val, Leu, Ile, Lys, Arg, Orn, Ser, Thr, —CN, —CONH₂, —COR′, —SO₂R′, —CONR′R″, —NHCOR′, OR′ and SO₂NR′R″; wherein said optionally substituted heterocyclyl is optionally singly or doubly substituted with substituents independently selected from the group consisting of C₁-C₆ alkyl, C₁-C₆ alkoxy, oxo, —OH, —Cl, —F, —NH₂, —NO₂, —CN, —COOH, and amidino; wherein R′ and R″ are each independently —H, C₁-C₈ alkyl, aryl, or heterocyclyl or R′ and R″ are combined to form a 4- to 8-membered ring, which ring is optionally singly or doubly substituted with substituents independently selected from the group consisting of C₁-C₆ alkyl, —C₁-C₆ alkoxy, —OH, —Cl, —F, —NH₂, —NO₂, —CN, —COOH and amidino; and R₂ is selected from the group consisting of —H, amidino, singly or doubly C₁-C₆ alkyl-substituted amidino, —CN, —CONH₂, —CONR′R″, —NHCOR′, —SO₂NR′R″ and —COOH; or

(ii) R₁ and R₂ taken together can form an optionally substituted 4- to 9-membered heterocyclic monocyclic or bicyclic ring moiety which is bonded to a single ring atom of the Y and Z-containing ring moiety;

(iii) R₁ and R₂ taken together with a single ring atom of the Y and Z-containing ring moiety can form an optionally substituted 4- to 8-membered heterocyclic ring moiety to form a spiro structure; or
(iv) R₁ and R₂ taken together with two or more adjacent ring atoms of the Y and Z-containing ring moiety can form an optionally substituted 4- to 9-membered heterocyclic monocyclic or bicyclic ring moiety fused to the Y and Z-containing ring moiety; wherein each of said optionally substituted 4-, 5-, 6-, 7-, 8- and 9-membered heterocyclic ring moieties comprising R₁ and R₂ is optionally singly or doubly substituted with substituents independently selected from the group consisting of C₁-C₆ alkyl, C₁-C₆ alkoxy, optionally substituted phenyl, oxo, —OH, —Cl, —F, —NH₂, —NO₂, —CN, —COOH, and amidino.

In one embodiment of the invention, the post-medical procedure inflammation is due to a medical procedure selected from the following procedures: appendectomy, open colorectal surgery, hernia repair, prostatectomy, colonic resection, gastrectomy, splenectomy, colectomy, colostomy, pelvic laparoscopy, tubal ligation, hysterectomy, vasectomy, cholecystectomy, colonoscopy, cystoscopy, hysteroscopy, cervical and endometrial biopsy. Other relevant medical procedures include ocular surgery procedures, such as for instance, canaloplasty, laser eye surgery and refractive surgery, such as lasik surgery (laser-assisted in-situ keratomileusis) and other corneal surgery including corneal replacement surgery, cataract surgery and retinal surgery. The term “post-medical procedure” pain and inflammation is used interchangeably with the term “post-surgical” pain and inflammation in this specification.

In still other embodiments the post-medical procedure inflammation may be due to an orthopedic procedure, such as for instance knee or shoulder arthroscopy, knee joint and hip joint replacement, carpel tunnel release, anterior cruciate ligament reconstruction, repair of femoral neck or ankle fracture, meniscectomy and laminectomy, to name but a few. Other orthopedic procedures addressable by the present invention will be immediately apparent to clinicians, surgeons and those of skill in the art.

### DETAILED DESCRIPTION

The D-isomer of an amino acid is specified by the prefix “D-” as in “D-Phe” which represents D-phenylalanine, the D-isomer of phenylalanine. Similarly, the L-isomer is specified by the prefix “L-” as in “L-Phe.” As used herein, D-Arg represents D-arginine, D-Har represents D-homoarginine, which has a side chain one methylene group longer than D-Arg, and D-Nar represents D-norarginine, which has a side chain one methylene group shorter than D-Arg. Similarly, D-Leu means D-leucine, D-Nle means D-norleucine, and D-Hle represents D-homoleucine. D-Ala means D-alanine, D-Tyr means D-tyrosine, D-Trp means D-tryptophan, and D-Tic means D-1,2,3,4-tetrahydroisoquinoline-3carboxylic acid. D-Val means D-valine and D-Met means D-methionine. D-Pro means D-proline, Pro-amide means the D- or L-form of proline amide. D-Pro amide represents D-proline with an amide formed at its carboxy moiety wherein the amide nitrogen may be alkyl substituted, as in —NRₐR_{b}, wherein Rₐ and R_{b} are each independently a C₁-C₆ alkyl group, or one of Rₐ and R_{b} is —H. Gly means glycine, D-Ile means D-isoleucine, D-Ser means D-serine, and D-Thr means D-threonine. (E)D-Ala means the D-isomer of alanine which is substituted by the substituent (E) on the β-carbon. Examples of such substituent (E) groups include tert-butyl, cyclopropyl, cyclobutyl, cyclopentyl, cyclohexyl, furyl, pyridyl, thienyl, thiazolyl and benzothienyl. Thus, cyclopentyl-D-Ala means the D-isomer of alanine which is substituted by cyclopentyl on the β-carbon. Similarly, D-Ala(2-thienyl) and (2-thienyl)D-Ala are interchangeable and both mean the D-isomer of alanine substituted at the β-carbon with thienyl that is attached at the 2-ring position.

As used herein, D-Nal means the D-isomer of alanine substituted by naphthyl on the β-carbon. D-2Nal means naphthyl substituted D-alanine wherein the attachment to naphthalene is at the 2-position on the ring structure and D-1Nal means naphthyl-substituted D-alanine wherein the attachment to naphthalene is at the 1-position on the ring structure. By (A)(A′)D-Phe is meant D-phenylalanine substituted on the phenyl ring with one or two substituents independently chosen from halo, nitro, methyl, halomethyl (such as, for example, trifluoromethyl), perhalomethyl, cyano and carboxamide. By D-(4-F)Phe is meant D-phenylalanine which is fluoro-substituted in the 4-position of the phenyl ring. By D-(2-F)Phe is meant D-phenylalanine which is fluoro-substituted in the 2-position of the phenyl ring. By D-(4-Cl)Phe is meant D-phenylalanine which is chloro substituted in the 4-phenyl ring position. By (α-Me)D-Phe is meant D-phenylalanine which is methyl substituted at the alpha carbon. By (α-Me)D-Leu is meant D-leucine which is methyl substituted at the alpha carbon.

The designations (B)₂D-Arg, (B)₂D-Nar, and (B)₂D-Har represent D-arginine, D-norarginine and D-homoarginine, respectively, each having two substituent (B) groups on the side chain. D-Lys means D-lysine and D-Hlys means D-homolysine. ζ-(B)D-Hlys, ε-(B)D-Lys, and ε-(B)₂-D-Lys represent D-homolysine and D-lysine each having the side chain amino group substituted with one or two substituent (B) groups, as indicated. D-Orn means D-ornithine and δ-(B)α-(B′)D-Orn means D-ornithine substituted with (B′) at the alpha carbon and substituted with (B) at the side chain 6-amino group.

D-Dap means D-2,3-diaminopropionic acid. D-Dbu represents the D-isomer of alpha, gamma-diamino butyric acid and (B)₂D-Dbu represents alpha, gamma-diamino butyric acid which is substituted with two substituent (B) groups at the gamma amino group. Unless otherwise stated, each of the (B) groups of such doubly substituted residues are independently chosen from H- and C₁-C₄-alkyl. As used herein, D-Amf means D-(NH₂CH₂—)Phe, i.e., the D-isomer of phenylalanine substituted with aminomethyl on its phenyl ring and D-4Amf represents the particular D-Amf in which the aminomethyl is attached at the 4-position of the ring. D-Gmf means D-Amf(amidino) which represents D-Phe wherein the phenyl ring is substituted with —CH₂NHC(NH)NH₂. Amd represents amidino, —C(NH)NH₂, and the designations (Amd)D-Amf and D-Amf(Amd) are also interchangeably used for D-Gmf. The designations Ily and Ior are respectively used to mean isopropyl Lys and isopropyl Orn, wherein the side chain amino group is alkylated with an isopropyl group.

Alkyl means an alkane radical which can be a straight, branched, and cyclic alkyl group such as, but not limited to, methyl, ethyl, propyl, isopropyl, cyclopropyl, butyl, t-butyl, sec-butyl, pentyl, cyclopentyl, hexyl, cyclohexyl, cyclohexylethyl. C₁ to C₈ alkyl refers to alkyl groups having between one and eight carbon atoms. Similarly, C₁-C₆ alkyl refers to alkyl groups having between one and six carbon atoms. Likewise, C₁-C₄ alkyl refers to alkyl groups having between one and four carbon atoms. By lower alkyl is meant C₁-C₆ alkyl. Me, Et, Pr, Ipr, Bu, and Pn are interchangeably used to represent the common alkyl groups: methyl, ethyl, propyl, isopropyl, butyl, and pentyl, respectively. Although the linkage for an alkyl group is typically at one end of an alkyl chain, the linkage may be elsewhere in the chain, e.g. 3-pentyl which may also be referred to as ethylpropyl, or 1-ethylprop-1-yl. Alkyl-substituted, such as C₁ to C₆ alkyl-substituted amidino, indicates that the relevant moiety is substituted with one or more alkyl groups.

Where a specified moiety is null, the moiety is absent and if such moiety is indicated to be attached to two other moieties, such two other moieties are connected by one covalent bond. Where a connecting moiety is shown herein as attached to a ring at any position on the ring, and attached to two other moieties, such as R₁ and R₂, in the case where the connecting moiety is specified to be null, then the R₁ and R₂ moieties can each be independently attached to any position on the ring.

The terms “heterocycle”, “heterocyclic ring” and “heterocyclyl” are used interchangeably herein and refer to a ring or ring moiety having at least one non-carbon ring atom, also called a heteroatom, which can be a nitrogen atom, a sulfur atom, or an oxygen atom. Where a ring is specified as having a certain number of members, the number defines the number of ring atoms without reference to any substituents or hydrogen atoms bonded to the ring atoms. Heterocycles, heterocyclic rings and heterocyclyl moieties can include multiple heteroatoms independently selected from nitrogen, sulfur, or oxygen atom in the ring. Rings can be substituted at any available position. For example, but without limitation, 6- and 7-membered rings are often substituted in the 4-ring position and 5-membered rings are commonly substituted in the 3-position, wherein the ring is attached to the peptide amide chain at the 1-ring position.

The term “saturated” means an absence of double or triple bonds and the use of the term in connection with rings describes rings having no double or triple bonds within the ring, but does not preclude double or triple bonds from being present in substituents attached to the ring.

The term “non-aromatic” in the context of a particular ring refers to an absence of aromaticity in that ring, but does not preclude the presence of double bonds within the ring, including double bonds which are part of an aromatic ring fused to the ring in question. Nor is a ring atom of a saturated heterocyclic ring moiety precluded from being double-bonded to a non-ring atom, such as for instance a ring sulfur atom being double-bonded to an oxygen atom substituent. As used herein, heterocycles, heterocyclic rings and heterocyclyl moieties also include saturated, partially unsaturated and heteroaromatic rings and fused bicyclic ring structures unless otherwise specified. A heterocycle, heterocyclic ring or heterocyclyl moiety can be fused to a second ring, which can be a saturated, partially unsaturated, or aromatic ring, which ring can be a heterocycle or a carbocycle.

Where indicated, two substituents can be optionally taken together to form an additional ring. Rings may be substituted at any available position. A heterocycle, heterocyclic ring and heterocyclyl moiety can, where indicted, be optionally substituted at one or more ring positions with one or more independently selected substituents, such as for instance, C₁-C₆ alkyl, C₃-C₈ cycloalkyl, C₁-C₆ alkoxy, halo C₁-C₆ alkyl, optionally substituted phenyl, aryl, heterocyclyl, oxo, —OH, —Cl, —F, —NH₂, —NO₂, —CN, —COOH and amidino. Suitable optional substituents of the phenyl substituent include for instance, but without limitation, one or more groups selected from C₁-C₃ alkyl, C₁-C₃ alkoxy, halo C₁-C₃ alkyl, oxo, —OH, —Cl, —F, —NH₂, —NO₂, —CN, —COOH and amidino.

D-Phe and substituted D-Phe are examples of a suitable amino acid for residue Xaa₁ in Formula I. The phenyl ring can be substituted at any of the 2-, 3- and/or 4-positions. Particular examples of permitted substitutions include, for instance, chlorine or fluorine at the 2- or 4-positions. Also the alpha-carbon atom may be methylated. Other equivalent residues which represent conservative changes to D-Phe can also be used. These include D-Ala(cyclopentyl), D-Ala(thienyl), D-Tyr and D-Tic. The residue at the second position, Xaa₂ can also be D-Phe or substituted D-Phe with such substitutions including a substituent on the 4-position carbon of the phenyl ring, or on both the 3- and 4-positions. Alternatively, Xaa₂ can be D-Trp, D-Tyr or D-alanine substituted by naphthyl. The third position residue, Xaa₃ can be any non-polar amino acid residue, such as for instance, D-Nle, D-Leu, (α-Me)D-Leu, D-Hle, D-Met or D-Val. However, D-Ala(cyclopropyl, cyclobutyl, cyclopentyl or cyclohexyl) or D-Phe can also be used as Xaa₃. The fourth position residue Xaa₄ can be any positively charged amino acid residue, such as for instance, D-Arg and D-Har, which can be optionally substituted with lower alkyl groups, such as one or two ethyl groups. Alternatively, D-Nar and any other equivalent residues can be used, such as, for instance, D-Lys or D-Orn (either of which can be w-amino group alkylated, for example by methyl or isopropyl groups, or methylated at the α-carbon group). Moreover, D-Dbu, D-4-Amf (which can be optionally substituted with amidino), and D-Hlys are also suitable amino acids at this position.

Compounds of the invention contain one or more chiral centers, each of which has two possible three-dimensional spatial arrangements (configurations) of the four substituents around the central carbon atom. These are known as “stereoisomers”, and more specifically as “enantiomers” (all chiral centers inverted) or “diastereoisomers” (two or more chiral centers, at least one chiral center remaining the same). In a specific embodiment of the invention, the amino acids which make up the tetrapeptide backbone, Xaa₁Xaa₂Xaa₃Xaa₄ are specified to be D-amino acids i.e., the opposite configuration to those generally found in mammals.

References to stereoisomers of the synthetic peptide amides of the invention in this specification relate to chiral centers other than the alpha carbons of the D-amino acids which make up Xaa₁-Xaa₄. Thus, stereoisomers of synthetic peptide amides that are embodiments of the invention wherein each of Xaa₁-Xaa₄ are specified to be D-amino acids, do not include L-amino acids or racemic mixtures of the amino acids at these positions. Similarly, reference to racemates herein concerns a center other than the alpha carbons of the D-amino acids which make up Xaa₁-Xaa₄. Chiral centers in the synthetic peptide amides of the invention for which a stereoisomer may take either the R or S configuration include chiral centers in the moiety attached to the carboxy-terminus of Xaa₄, and also chiral centers in any amino acid side chain substituents of Xaa₁-Xaa₄.

As used herein, “effective amount” or “sufficient amount” of the peripherally-restricted kappa receptor agonists of the invention such as the synthetic peptide amides refers to an amount of the compound as described herein that may be therapeutically effective to inhibit, prevent, or treat a symptom of a particular disease, disorder, condition, or side effect. As used herein, a “reduced dose” of a mu opioid agonist analgesic compound refers to a dose which when used in combination with a kappa opioid agonist, such as a synthetic peptide amide of the invention, is lower than would be ordinarily provided to a particular patient, for the purpose of reducing one or more side effects of the compound. The dose reduction can be chosen such that the decrease in the analgesic or other therapeutic effect of the compound is an acceptable compromise in view of the reduced side effect(s), where the decrease in analgesic or other therapeutic effects of the mu opioid agonist analgesic are wholly or at least partially offset by the analgesic or other therapeutic effect of the synthetic peptide amide of other peripherally-restricted kappa receptor agonist of the invention.

Co-administration of a mu opioid agonist analgesic compound with a synthetic peptide amide or other peripherally-restricted kappa receptor agonist of the invention also permits incorporation of a reduced dose of the peripherally-restricted kappa receptor agonist or synthetic peptide amide and/or the mu opioid agonist analgesic compound to achieve the same therapeutic effect as a higher dose of the synthetic peptide amide/other peripherally-restricted kappa receptor agonist or the mu opioid agonist analgesic compound if administered alone.

As used herein, “pharmaceutically acceptable” refers to compounds, materials, compositions, and/or dosage forms which are, within the scope of sound medical judgment, suitable for contact with the tissues of human beings and animals without severe toxicity, irritation, allergic response, or other complications, commensurate with a benefit-to-risk ratio that is reasonable for the medical condition being treated.

As used herein, “dosage unit” refers to a physically discrete unit suited as unitary dosages for a particular individual or condition to be treated. Each unit may contain a predetermined quantity of active synthetic peptide amide or other peripherally-restricted kappa receptor agonist compound(s) calculated to produce the desired therapeutic effect(s), optionally in association with a pharmaceutical carrier. The specification for the dosage unit forms may be dictated by (a) the unique characteristics of the active compound or compounds, and the particular therapeutic effect to be achieved, and (b) the limitations inherent in the art of compounding such active compound or compounds. The dosage unit is often expressed as weight of compound per unit body weight, for instance, in milligrams of compound per kilogram of body weight of the subject or patient (mg/kg). Alternatively, the dosage can be expressed as the amount of the compound per unit body weight per unit time, (mg/kg/day) in a particular dosage regimen. In a further alternative, the dosage can be expressed as the amount of compound per unit body surface area (mg/m²) or per unit body surface area per unit time (mg/m²/day). For topical formulations, the dosage can be expressed in a manner that is conventional for that formulation, e.g., a one-half inch ribbon of ointment applied to the eye, where the concentration of compound in the formulation is expressed as a percentage of the formulation.

As used herein, a “pharmaceutically acceptable salt” refers to a derivative of a compound wherein the parent compound is modified by making an acid or a base salt thereof. Examples of pharmaceutically acceptable salts include, but are not limited to, mineral or organic acid salts of basic residues such as amines; alkali or organic salts of acidic residues such as carboxylic acids and the like. The pharmaceutically acceptable salts include the conventional non-toxic salts or the quaternary ammonium salts of the parent compound formed, for example, from non-toxic inorganic or organic acids. For instance, such conventional non-toxic salts include those derived from inorganic acids such as hydrochloric, hydrobromic, sulfuric, sulfamic, phosphoric, nitric acids and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic acids, and the like.

These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine. Thus, a pharmaceutically acceptable salt of a synthetic peptide amide or other peripherally-restricted kappa receptor agonist can be formed from any such peptide amide having either acidic, basic or both functional groups. For example, a peptide amide having a carboxylic acid group, may in the presence of a pharmaceutically suitable base, form a carboxylate anion paired with a cation such as a sodium or potassium cation. Similarly, a peptide amide having an amine functional group may, in the presence of a pharmaceutically suitable acid such as HCl, form a salt.

An example of a pharmaceutically acceptable solvate of a synthetic peptide amide or other peripherally-restricted kappa receptor agonist is a combination of a synthetic peptide amide or other peripherally-restricted kappa receptor agonist with solvent molecules which yields a complex of such solvent molecules in association with the synthetic peptide amide or other peripherally-restricted kappa receptor agonist. Combinations of a drug and propylene glycol (1,2-propanediol) have been used to form pharmaceutical drug solvates. See for example U.S. Pat. No. 3,970,651. Other suitable solvates are hydrates of drug compounds. Such hydrates include hydrates which either have comparable activity or hydrates which are converted back to the active compound following administration. A pharmaceutically acceptable N-oxide of a synthetic peptide amide or other peripherally-restricted kappa receptor agonist is such a compound that contains an amine group wherein the nitrogen of the amine is bonded to an oxygen atom.

A pharmaceutically acceptable crystalline, isomorphic crystalline or amorphous form of a synthetic peptide amide or other peripherally-restricted kappa receptor agonist of the invention can be any crystalline or non-crystalline form of a pharmaceutically acceptable acidic, basic, zwitterionic, salt, hydrate or any other suitably stable, physiologically compatible form of the synthetic peptide amide or other peripherally-restricted kappa receptor agonist according to the invention.

The synthetic peptide amides or other peripherally-restricted kappa receptor agonists of the invention can be incorporated into pharmaceutical compositions. The compositions can include an effective amount of the synthetic peptide amide or other peripherally-restricted kappa receptor agonist in a pharmaceutically acceptable diluent, excipient or carrier. Conventional excipients, carriers and/or diluents for use in pharmaceutical compositions are generally inert and make up the bulk of the preparation.

In one embodiment, the present invention provides a method for reducing post medical procedure pain, post medical procedure inflammation, or both in a mammalian subject, the method comprising administering an effective amount of a peripherally restricted kappa opioid receptor agonist to the subject prior to the medical procedure that normally causes post-medical procedure pain, post-medical procedure inflammation, or both and thereby reducing the post-medical procedure pain and/or the post-medical procedure inflammation experienced by the subject; wherein the peripherally restricted kappa opioid receptor agonist has the structure of formula I:

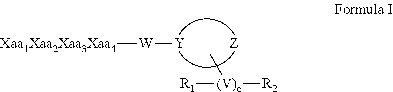

wherein the Y and Z-containing ring moiety is a six or seven membered ring having a single ring heteroatom and e is zero, then R₁ is not —OH, and R₁ and R₂ are not both —H.

In another embodiment when the Y and Z-containing ring moiety of formula I is a six membered ring having two ring heteroatoms, both Y and Z are N and W is null, then -(V)ₑR₁R₂ is attached to a ring atom other than Z; and when e is zero, then R₁ and R₂ are not both —H.

In another embodiment of the synthetic peptide useful in the practice of the present invention, Xaa₁Xaa₂ of formula I is D-Phe-D-Phe, Xaa₃ is D-Leu or D-Nle and Xaa₄ is selected from the group consisting of (B)₂D-Arg, D-Lys, (B)₂D-Har, ζ-(B)D-Hlys, D-Dap, ε-(B)D-Lys, ε-(B)₂-D-Lys, D-Amf, amidino-D-Amf, γ-(B)₂D-Dbu and δ-(B)₂α-(B′)D-Orn.

In another embodiment of the synthetic peptide of formula I useful in the practice of the present invention, Xaa₄ is selected from D-Lys, (B)₂D-Har, ε-(B)D-Lys and ε-(B)₂-D-Lys.

In still another embodiment of the synthetic peptide of formula I useful in the practice of the present invention, W is null, Y is N and Z is C.

In another embodiment of the synthetic peptide of formula I, the Y and Z-containing ring moiety is a six-membered saturated ring comprising a single ring heteroatom.

In another embodiment of the synthetic peptide of formula I useful in the practice of the present invention, the groups R₁ and R₂ taken together with zero, one or two ring atoms of the Y and Z-containing ring moiety comprise a monocyclic or bicyclic 4-9 membered heterocyclic ring moiety.

In still another embodiment of the synthetic peptide of formula I useful in the practice of the present invention, the moiety e is zero and the groups R₁ and R₂ are each bonded directly to the same ring atom, R₁ is H, OH, —NH₂, —COOH, —CH₂COOH, C₁-C₃ alkyl, amidino, C₁-C₃ alkyl-substituted amidino, dihydroimidazole, D-Pro, D-Pro amide, or CONH₂ and R₂ is H, —COOH, or C₁-C₃ alkyl.

In another embodiment of the synthetic peptide of formula I useful in the practice of the present invention, the moiety:

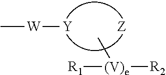

is selected from the following groups:

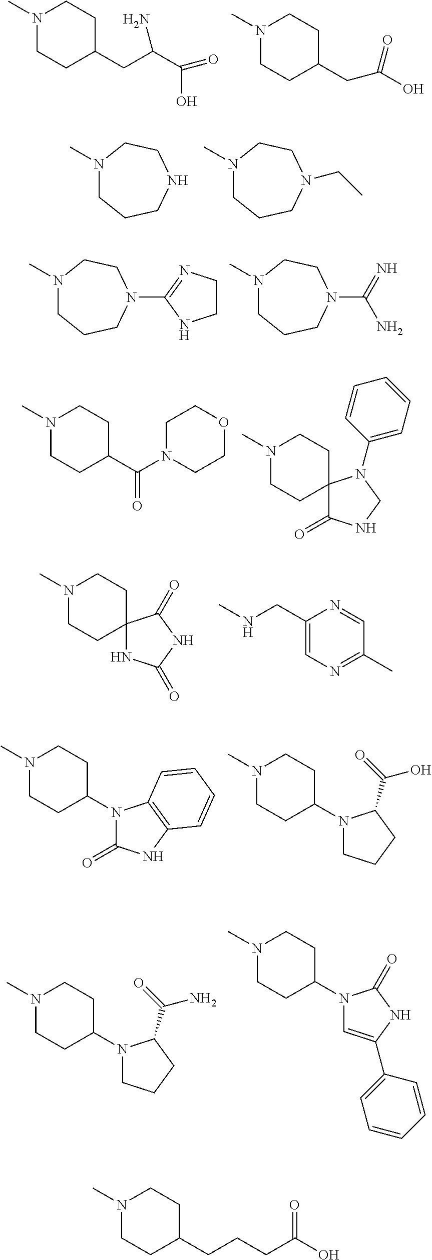

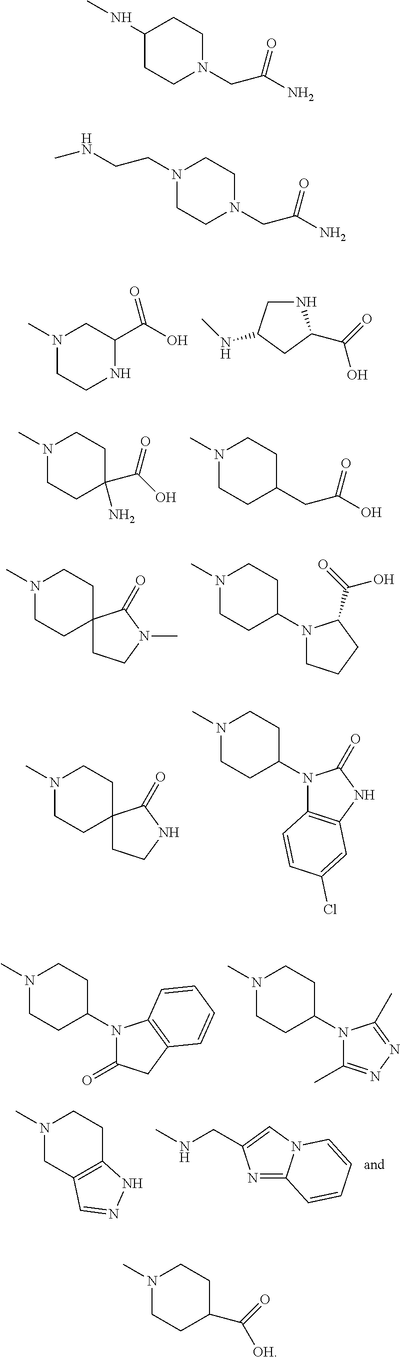

In another embodiment of the synthetic peptide of formula I useful in the practice of the present invention, Xaa₁Xaa₂ is D-Phe-D-Phe, Xaa₃ is D-Leu or D-Nle and Xaa₄ is selected from (B)₂D-Arg, D-Lys, (B)₂D-Har, ζ-(B)D-Hlys, D-Dap, ε-(B)D-Lys, ε-(B)₂-D-Lys, D-Amf, amidino-D-Amf, γ-(B)₂D-Dbu and δ-(B)₂α-(B′)D-Orn.

In another embodiment of the synthetic peptide useful in the practice of the present invention is CR845 having the structure: D-Phe-D-Phe-D-Leu-D-Lys-[ω(4-aminopiperidine-4-carboxylic acid)]-OH:

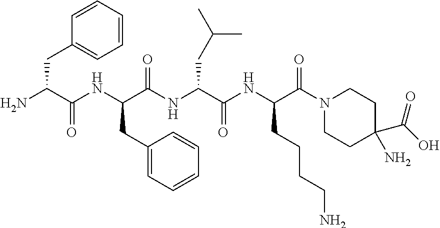 [SMILES: CC(C)C[C@@H](NC(=O)[C@@H](Cc1ccccc1)NC(=O)[C@H](N)Cc1ccccc1)C(=O)N[C@H](CCCCN)C(=O)N1CCC(N)(C(=O)O)CC1]

Asimadoline, a selective, non-peptidic kappa-opioid receptor agonists is also useful I methods according to the present invention for pretreatment prior to surgery in order to reduce post-surgical pain and inflammation. Asimadoline has the diarylacetamide structure shown below:

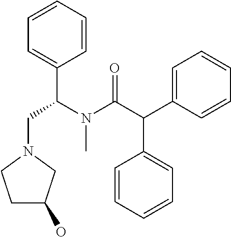 [SMILES: CN(C(=O)C(c1ccccc1)c1ccccc1)[C@H](CN1CC[C@H](O)C1)c1ccccc1]

(N-[(1S)-2-[(3S)-3-hydroxypyrrolidin-1-yl]-1-phenylethyl]-N-methyl-2,2-diphenylacetamide).

Nalfurafine (also known as AC-820, TRK-820) is a kappa opioid receptor agonist marketed as a treatment for uremic pruritus in hemodialysis patients. Nalfurafine is another kappa opioid receptor agonist useful according to the present invention for pretreatment prior to surgery in order to reduce post-surgical pain and inflammation.

Nalfurafine is (2E)-N-[(5α,6β)-17-(cyclopropylmethyl)-3,14-dihydroxy-4,5-epoxymorphinan-6-yl]-3-(3-furyl)-N-methylacrylamide, and has the following chemical structure:

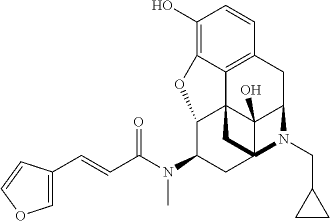

In one embodiment of the invention, the method further includes administering another dose of the peripherally restricted kappa opioid receptor agonist to the subject during or after the medical procedure.

In another embodiment of the method of the invention, the relief from post-medical procedure pain is such that the subject self administers little or no patient controlled analgesia (PCA) after surgery and/or during recovery.

In another embodiment of the method of the invention, the relief from post-medical procedure inflammation is accompanied by relief from pruritis during the period of recovery from the medical procedure.

In one embodiment of the invention, the peripherally restricted kappa opioid receptor agonist useful in the practice of the present invention is a non-narcotic analgesic.

In another embodiment of the method of the invention, the peripherally restricted kappa opioid receptor agonist is administered by a route of injection selected from the group consisting of subcutaneous injection, intravenous injection, intraperitoneal injection, intra-articular injection, intramuscular injection or intra-ocular injection.

Inflammation is a complex biological response of vascular tissues to harmful stimuli, such as pathogens, damaged cells, or irritants. Inflammation can be acute or chronic, and is often characterized by fever in mammals. Inflammation is a protective response to an inflammatory stimulus and initiates wound-healing processes. Interleukin-6 (IL6) is an anti-inflammatory cytokine. The level of C-reactive protein (CRP) in blood rise in the acute phase of inflammation. Therefore, the level of CRP is a useful indicator of an acute inflammatory response. The level of C-reactive protein can be determined by commercially available ELISA assays. IL6 and CRP have been as clinical markers of inflammation, see for instance Esme et al. (2011) Effects of Flurbiprofen on CRP, TNF-, IL6 and Postoperative Pain of Thoractomy; Int. J. Med. Sci. vol. 8(3): pp 216-221.

The anti-inflammatory cytokine, Interleukin-6 (IL6) is assayed as follows: MSD® Cytokine Assays are used to measure IL6 in 96-well MULTI-ARRAY® or MULTISPOT® plates. The assays employ a sandwich immunoassay format where capture antibodies are coated in a single spot, or in a patterned array, on the bottom of the wells of a MULTIARRAY® or MULTI-SPOT® plate.

The sample and a solution containing the labeled detection antibody-anti-IL6 antibody labeled with an electrochemiluminescent compound, MSD SULFO-TAG™ label—are added over the course of one or more incubation periods. The IL6 in the sample binds to capture antibody immobilized on the working electrode surface; recruitment of the labeled detection antibody by bound cytokine completes the sandwich. The user adds an MSD read buffer that provides the appropriate chemical environment for electrochemiluminescence and loads the plate into an MSD SECTOR® instrument for analysis. Inside the SECTOR® instrument, a voltage applied to the plate electrodes causes the labels bound to the electrode surface to emit light. The instrument measures intensity of emitted light to provide a quantitative measure of IL6 present in the sample.

### EXAMPLES

### Example 1

### First in Man Clinical Trial of a Peripherally-Restricted Non-Narcotic Kappa Receptor Agonist

This trial was a phase 2 multi-center, double-randomized, double-blind, placebo-controlled study was conducted to evaluate the analgesic efficacy and safety of intravenous CR845 dosed preoperatively and postoperatively in patients undergoing a laparoscopic hysterectomy. The trial was conducted under an investigational new drug application (IND) filed with the US Food and Drug Administration (FDA).

This phase 2 trial multi-center, double-randomized, double-blind, placebo-controlled study was conducted in 22 sites in the United States. All clinical procedures were approved by the relevant Institutional Review Board (IRB) in compliance with the applicable laws and regulations of the US. The study was initiated up to fourteen (14) days preoperatively and the in-hospital period was approximately twenty-four (24) hours for each patient and follow up was conducted within seven (7) days of discharge from the hospital.

The all-D-tetrapeptide amide, CR845 is a peripherally-restricted kappa-opioid receptor agonist, in Phase 2 of clinical development for the treatment of acute pain. Thus far, CR845 has been shown to have analgesic and morphine-sparing effects in a proof of concept clinical study when administered after surgery (single intravenous dose) (Menzaghi et al., IASP 2010). In addition, CR845 appeared to be better tolerated in comparison to other known kappa agonists, likely due to its limited CNS penetration.

The aim of this clinical trial was to evaluate the analgesic efficacy and safety of intravenous (IV) CR845 dosed preoperatively and postoperatively in female patients undergoing an elective laparoscopic hysterectomy. Secondary objectives were: 1. To evaluate the efficacy of CR845 compared to placebo in reducing pain following laparoscopic hysterectomy: (a) to determine the time specific visual analog pain score (VAS) difference, summed pain intensity differences over all time periods (SPIDs) and pain relief, etc. and (b) to evaluate the proportion of patents that were randomized to receive preoperative CR845 and received no rescue medication postoperatively, compared to those who received placebo preoperatively and did not receive any rescue medication; and 2. To evaluate the safety and tolerability of CR845.

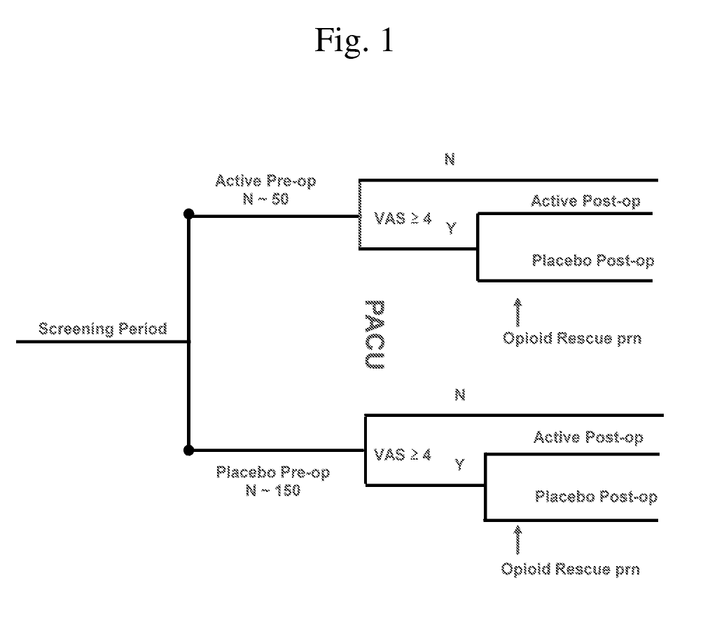

This Phase 2, double-randomized, double-blind, placebo-controlled, parallel group study was conducted in approximately 200 female subjects, aged 18 to 65, across 22 US clinical sites. All subjects were administered a 15-minute IV infusion of 0.04 mg/kg CR845 or matching placebo before surgery in a 1:1 ratio. After surgery and upon reaching a pain intensity baseline level of at least 4 on a 10 cm visual analog scale (VAS), subjects were also re-randomized in a 1:1 ratio to receive a second IV infusion of either 0.04 mg/kg of CR845 or placebo. All doubly-randomized patients were thereby randomized to receive one of four possible treatment regimens: placebo-placebo, placebo-CR845, CR845-placebo, or CR845-CR845 (See FIG. 1). Pain intensity and pain relief scores were assessed up to 24 hours following the post-surgery infusion. Patients were allowed IV morphine for rescue at any point during the post-operative period, if requested.

The main efficacy endpoints were the total morphine consumption in the first 24 hours in patients who were re-randomized in the postoperative period and the efficacy of CR845 in reducing pain intensity including time specific VAS difference; summed pain intensity differences (SPIDs); area under the curve (AUC); pain relief; and the proportion of patients that were randomized to receive preoperative CR845 with no rescue medication postoperatively. The safety endpoints included the incidence of adverse events, physical examination, vital signs, 12-lead ECG, clinical laboratory evaluations, and cumulative fluid balance.

### Example 2

### Relative Levels of Reduction of Post-Operative Pain (SPIDs)

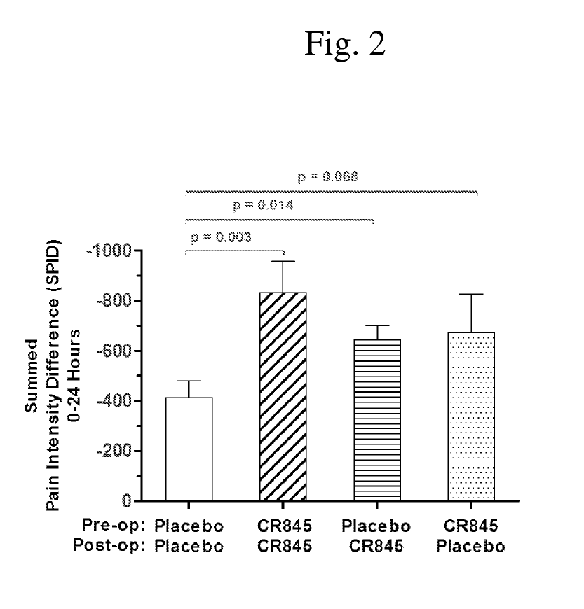

FIG. 2 shows the relative levels of reduction of post-operative pain over the first 24 hours after surgery as demonstrated by the summed pain intensity difference (SPID) summed over all of the time points. Patients receiving only placebo showed the least pain reduction and those receiving CR845 both before and after surgery showed the greatest reduction in the SPID pain score (more than twice the reduction over 24 hours as experienced by the patients receiving only placebo). Patients receiving one dose of CR845, whether before or after surgery exhibited an intermediate level of reduction in pain SPID scores (i.e. more relief than seen in patients receiving only placebo, but less than the relief experienced by those patients receiving CR845 before surgery and also after surgery).

### Example 2

### Relative Levels of Reduction of Post-Operative Pain (PIDs)

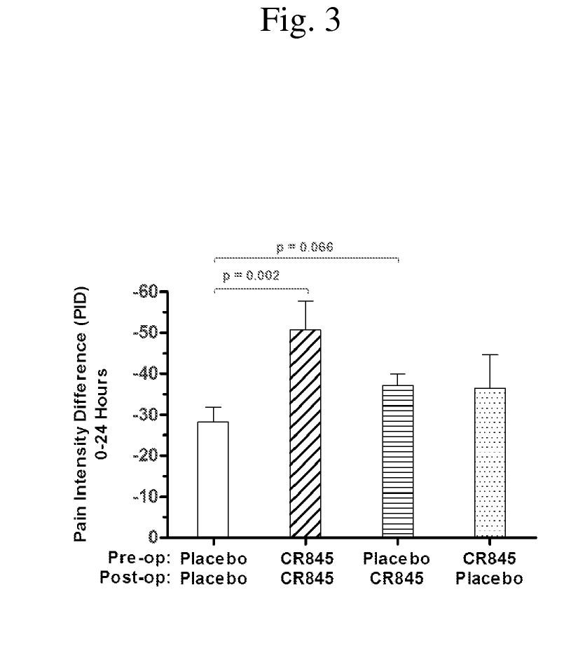

FIG. 3 shows the relative levels of reduction of post-operative pain over the first 24 hours after surgery as demonstrated by the pain intensity difference (PID) at each time point. As shown above for the summed pain intensity difference, patients receiving only placebo showed the least pain reduction and those receiving CR845 both before and after surgery showed the greatest reduction in the SPID pain score (twice the reduction over 24 hours as experienced by the patients receiving only placebo). Patients receiving one dose of CR845, whether before or after surgery exhibited an intermediate level of reduction in pain PID scores (i.e. more relief than seen in patients receiving only placebo, but less than the relief experienced by those patients receiving CR845 before surgery and also after surgery).

### Example 3

### Morphine Self-Administered by the Patients

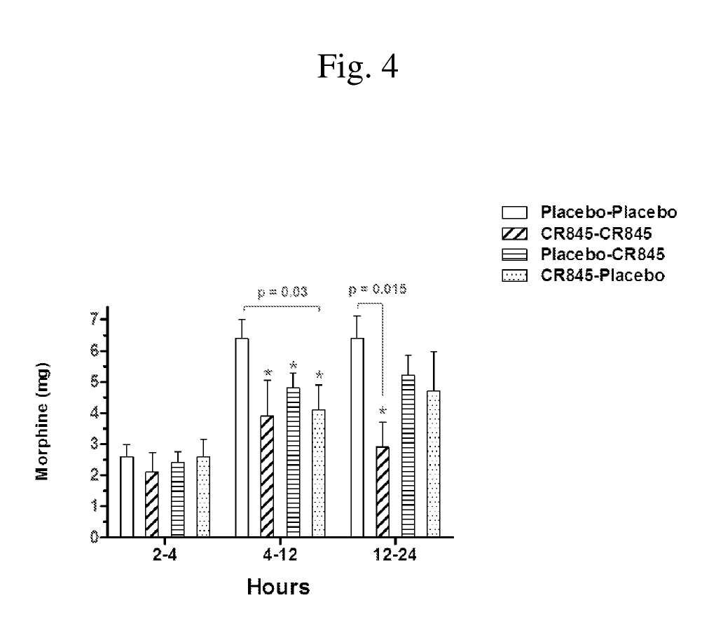

FIG. 4 shows the amounts of morphine (in milligrams) self administered by the patients in each group: placebo-placebo, placebo-CR845, CR845-placebo, and CR845-CR845 in the interval 2-4 hours, 4-12 hours and 12-24 hours post surgery. Patients receiving only placebo self-administered the most intrathecally delivered morphine (mITT), more than doubling the dose self administered from 2-4 hours during each of the 4-12 hour and 12-24 hour periods. By contrast, those patients receiving two doses of CR845 self-administered the least morphine in each time period.

### Example 4

### Evaluations of Pain Relief Assessed by Patients

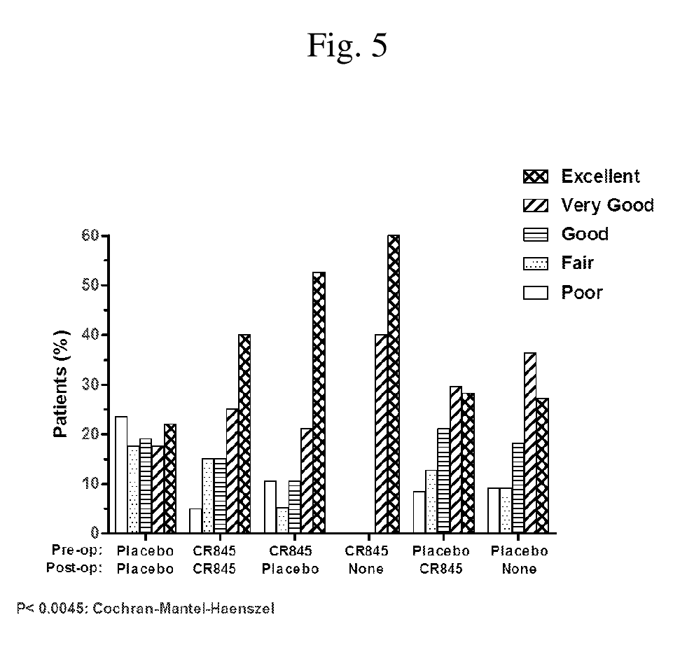

FIG. 5 shows the evaluations of pain relief as assessed by the patients themselves. The four patient groups are those receiving pre- and post-operatively respectively:
(a) Placebo-Placebo;
(b) CR845-CR845;
(c) Placebo-CR845; and
(d) CR845-Placebo.

The Placebo-Placebo group (i.e receiving inactive placebo instead of the peripherally-restricted kappa opioid receptor agonist) showed the lowest pain intensity difference (i.e. the least pain relief) and the group receiving CR845 before and after surgery showed the highest pain intensity difference (i.e. most pain relief) as judged by PID score.

### Example 4

### Global Evaluations of Pain Relief Assessed by Patients

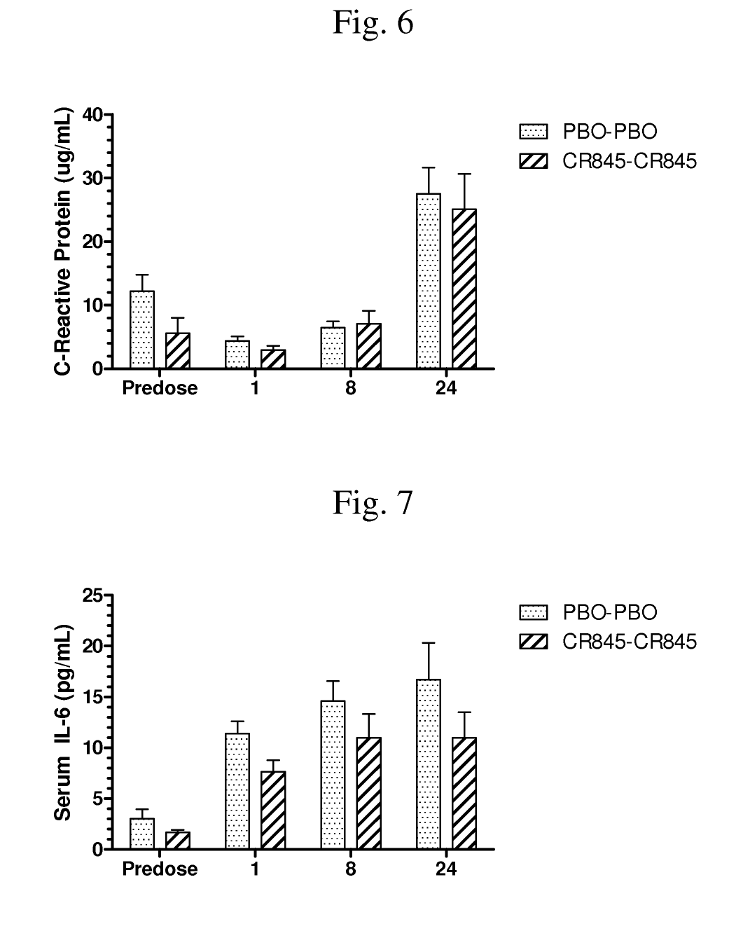

FIG. 6 shows the overall evaluations of pain relief as assessed by the patients themselves. The six patient groups are those receiving pre- and post-operatively respectively:
(a) Placebo-Placebo; (71 patients)
(b) CR845-CR845; (20 patients)
(c) CR845-Placebo; (19 patients)
(d) CR845-None; (5 patients)
(e) Placebo-CR845; (71 patients) and
(f) Placebo-None; (11 patients).

The highest percentage of patients giving an assessment of the overall evaluation of the pain relief as “excellent” were in the CR845 pretreatment groups, i.e. groups (b), (c) and (d). These data also show that groups pre-treated with CR845 were consistently evaluated as very good or excellent by the highest percentage of patients. The above-described clinical data demonstrate that CR845 represents an effective novel therapeutic class useful for pretreatment of postoperative pain and inflammation.

### Example 5

### Assay of IL6 in Patient Blood Samples

The anti-inflammatory cytokine, Interleukin-6 (IL6) is assayed using the MSD® Cytokine Assays to measure IL6 in 96-well MULTI-ARRAY® or MULTISPOT® plates (Meso Scale Discovery, Gaithersburg, Md.) according to the manufacturer's instructions. The assays employ a sandwich immunoassay format where capture antibodies are coated in a single spot, or in a patterned array, on the bottom of the wells of a MULTIARRAY® or MULTI-SPOT® plate.

The serum sample and a solution containing the labeled detection antibody-anti-IL6 antibody labeled with an electrochemiluminescent compound, MSD SULFO-TAG™ label—are added over the course of one or more incubation periods. The IL6 in the sample binds to capture antibody immobilized on the working electrode surface; recruitment of the labeled detection antibody by bound cytokine completes the sandwich. The user adds an MSD read buffer that provides the appropriate chemical environment for electrochemiluminescence and loads the plate into an MSD SECTOR® instrument for analysis. Inside the SECTOR® instrument, a voltage applied to the plate electrodes causes the labels bound to the electrode surface to emit light. The instrument measures intensity of emitted light to provide a quantitative measure of IL6 present in the sample.

Calibrators are run in duplicate to generate a standard curve. The standard curve is modeled using least squares fitting algorithms so that signals from samples with known levels of IL6 can be used to calculate the concentration of analyte in the sample. The assays have a wide dynamic range (3-4 logs) which allows accurate quantitation in many samples without the need for dilution. The MSD DISCOVERY WORKBENCH® analysis software utilizes a 4-parameter logistic model (or sigmoidal dose-response) and includes a 1/Y2 weighting function. The weighting function is important because it provides a better fit of data over a wide dynamic range, particularly at the low end of the standard curve.

### Example 6

### Assay of C-Reactive Protein in Patient Blood Samples

The Invitrogen Hu CRP kit is a solid phase sandwich Enzyme Linked-Immuno-Sorbent Assay (ELISA) useful for the determination of CRP levels in a sample and was used according to the manufacturer's instructions. Alternatively, CRP levels were determined using the C-Reactive Protein High Sensitivity Test by Roche Diagnostics according to the manufacturer's instructions.

Briefly, a highly purified antibody is coated onto the wells of the microtiter strips provided. During the first incubation, standards of known Hu CRP content, controls, and unknown samples were pipetted into the coated wells. After washing, biotinylated second antibody was added. After another washing, Streptavidin-Peroxidase (enzyme) was added. This binds to the biotinylated antibody to complete the four-member sandwich. After a third incubation and washing to remove all the unbound enzyme, a substrate solution was added, which was acted upon by the bound enzyme to produce color. The intensity of this colored product is directly proportional to the concentration of Hu CRP present in the original specimen. Results are shown in FIG. 6. Levels of CRP appeared to be comparable between patients treated with CR845 pre- and post-surgically versus placebo. However, it should be noted that CRP is produced by the liver not by immune cells and so may not be a good marker for surgically induced inflammation.

### Example 7

### Thirteen-Plex Assay of Cytokines in Patient Serum Samples

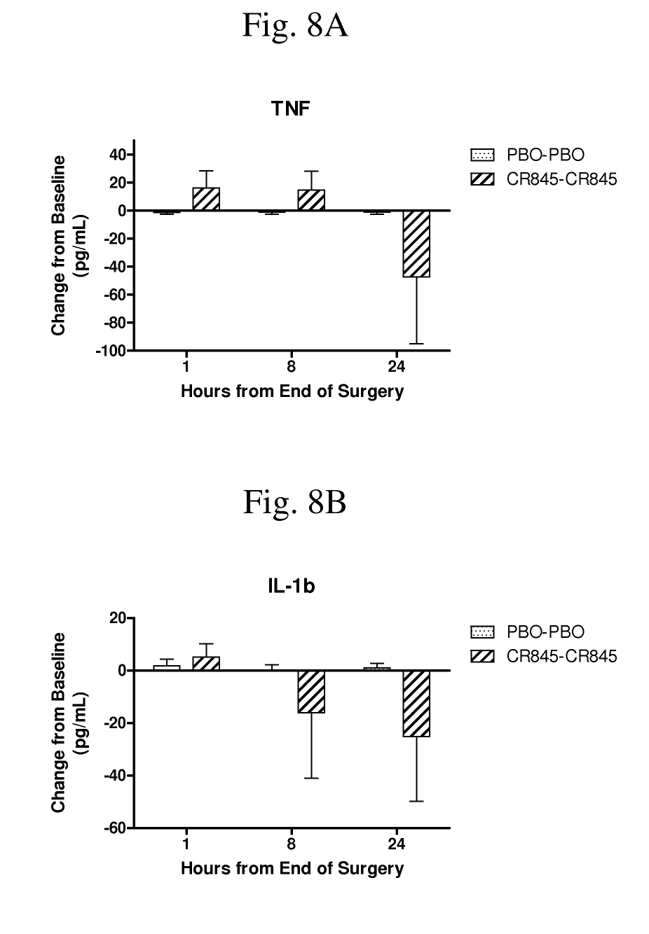

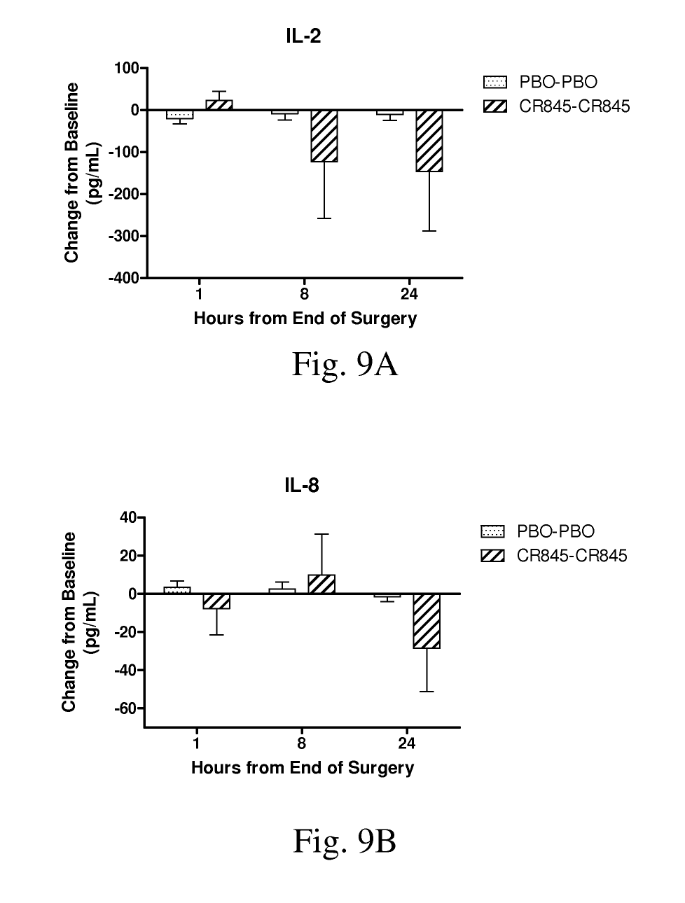

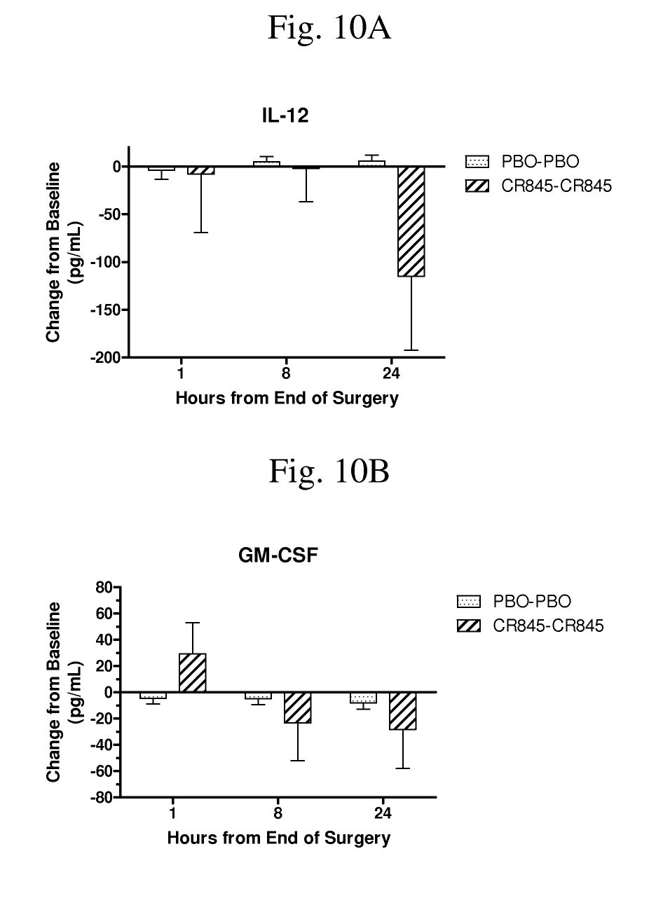

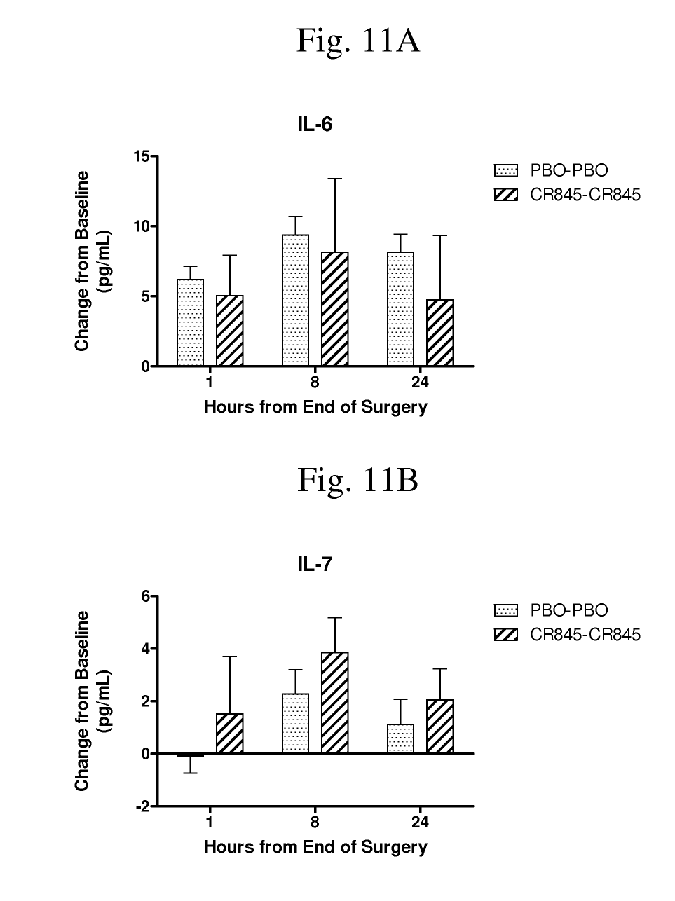

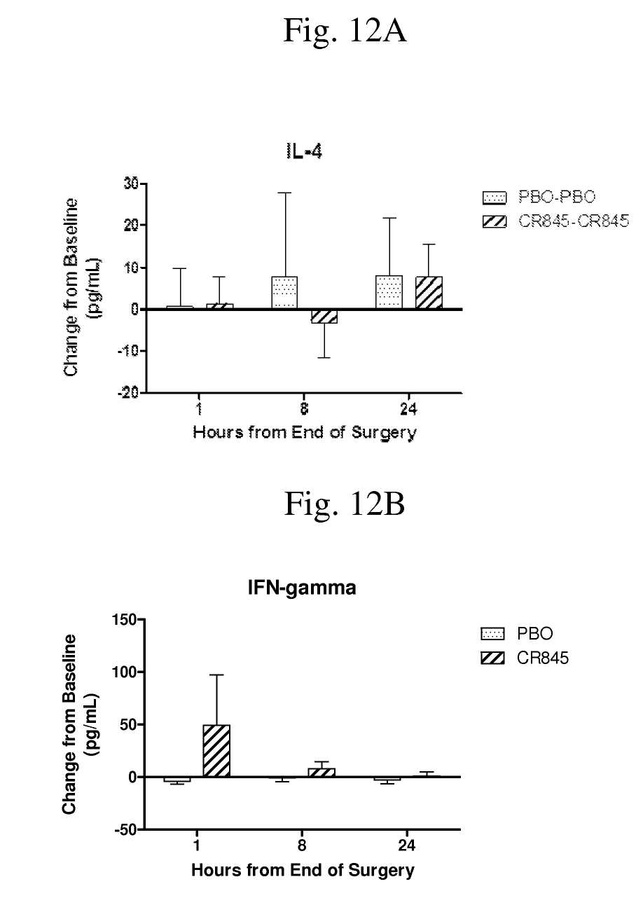

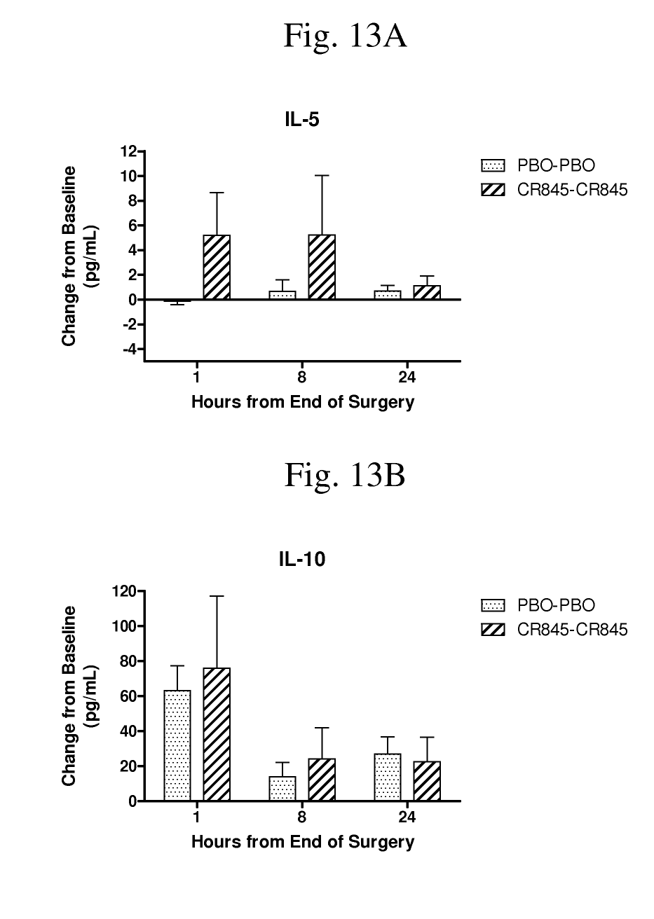

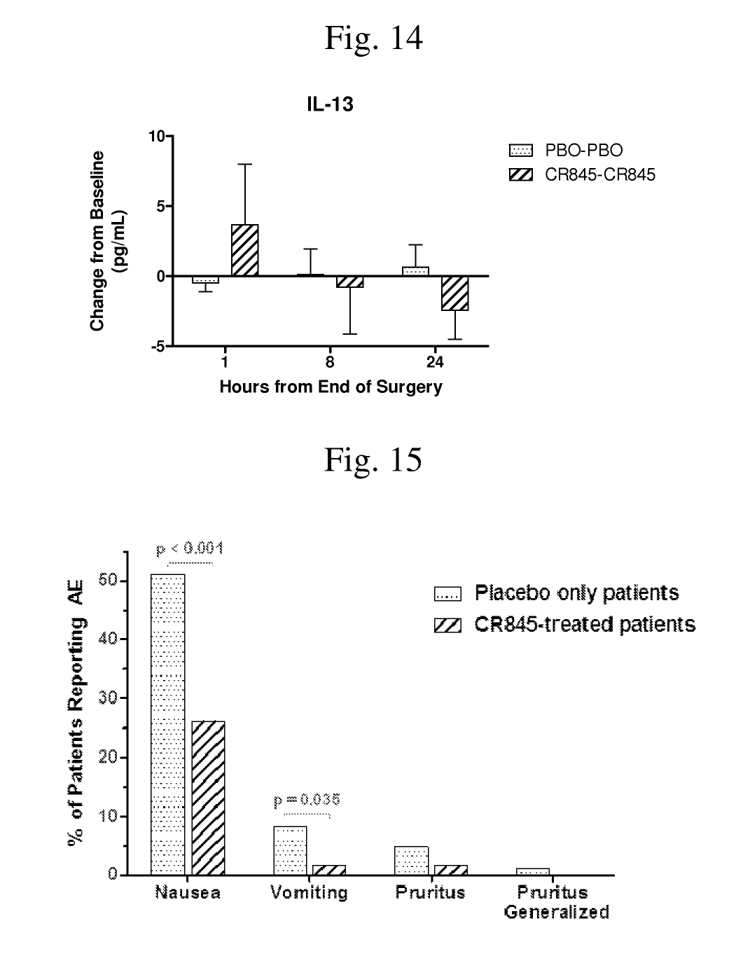

The Human Cytokine MILLIPLEX MAP assay panel with the Luminex® xMAP® microsphere technology optimized format (Millipore, St. Charles, Mo.) was used to measure serum concentrations of a panel of 13 cytokines: IL-1β, IL-2, IL-4, IL-5, IL-6, IL-7, IL-8, IL-10, IL-12(p70), IL-13, GM-CSF, IFNγ, and TNFα were quantified in human serum samples according to the manufacturer's instructions. Samples were serum samples taken at 1 hour, 8 hours and 24 hours post laparoscopic surgery in a Phase IIb clinical trial in which subjects were administered CR845 or placebo as shown in FIGS. 6-14.

The method uses Luminex® proprietary techniques to internally color-code microspheres with two fluorescent dyes. Assays depend on a hundred distinctly colored microsphere sets, each of which is coated with a specific capture antibody. The cytokine in the test sample is captured by the microspheres, and a biotinylated detection antibody is used as detection agent. The reaction mixture is then incubated with Streptavidin-PE conjugate, the reporter molecule, to complete the reaction on the surface of each microsphere. The microspheres are then passed rapidly through a laser which excites the internal dyes marking the microsphere set. A second laser excites PE, the fluorescent dye on the reporter molecule. A high-speed digital-signal processor is used to identify each individual microsphere and quantify the result of its bioassay based on fluorescent reporter signals. Multiple results were thus obtained from each sample.

Changes in cytokine levels were detected in most cases (see FIGS. 6-14), however, changes in cytokine levels that occurred between the three sampling times or even before 1 hour or after 24 hours post surgery would not have been detected.

### Example 8

### Adverse Events in Subjects Treated with CR845 or Placebo

Adverse events (nausea, vomiting and pruritus) occurring in the first 24 hours post laparoscopic surgery are shown in FIG. 15. Data are from a Phase IIb clinical trial in which subjects were administered CR845 or placebo. Generalized pruritus was distinguished from local pruritus events and shown separately in FIG. 15. Adverse events were lower in all CR845 treated patient groups as compared with placebo treated patients.

The specifications of each of the U.S. patents, allowed and published patent applications, and the texts of the literature references cited in this specification are herein incorporated by reference in their entireties. In the event that any definition or description contained found in one or more of these references is in conflict with the corresponding definition or description herein, then the definition or description disclosed herein is intended.

The examples provided herein are for illustration purposes only and are not intended to limit the scope of the invention, the full breadth of which will be readily recognized by those of skill in the art.

1. A method for reducing kappa opioid receptor-associated inflammation in a mammalian subject, the method comprising administering an effective amount of a peripherally-restricted kappa opioid receptor agonist to the subject.

2. The method according to claim 1, wherein the administering of the peripherally restricted kappa opioid receptor agonist causes a reduction in the level of one or more pro-inflammatory cytokines and/or the increase in an level of one or more anti-inflammatory cytokines in the blood of the subject.

3. The method according to claim 2, wherein the one or more pro-inflammatory cytokines are selected from the group consisting of IL-1β, IL-2, IL-4 IL-6, IL-7, IL-8, IL-12, GM-CSF, TNFα and IFNγ.

4. The method according to claim 2, wherein the one or more anti-inflammatory cytokines are selected from the group consisting of IL-5, IL-10 and IL-13.

5. The method according to claim 1, wherein the inflammation is due to an inflammatory disease or condition.

6. The method according to claim 1, wherein the inflammation is due to a medical procedure.

7. The method according to claim 1, wherein the inflammation is due to a physical insult.

8. The method according to claim 5, wherein the inflammatory disease or condition is selected from the group consisting of sinusitis, rheumatoid arthritis tenosynovitis, bursitis, tendonitis, lateral epicondylitis, adhesive capsulitis, osteomyelitis, osteoarthritic inflammation, inflammatory bowel disease (IBD), irritable bowel syndrome (IBS), ocular inflammation, otitic inflammation and autoimmune inflammation.

9. The method according to claim 6, wherein the inflammation is due to a medical procedure selected from the group consisting of appendectomy, open colorectal surgery, hernia repair, prostatectomy, colonic resection, gastrectomy, splenectomy, colectomy, colostomy, pelvic laparoscopy, tubal ligation, hysterectomy, vasectomy, cholecystectomy, colonoscopy, cystoscopy, hysteroscopy, cervical and endometrial biopsy.

10. The method according to claim 6, wherein the inflammation is due to a physical insult selected from the group consisting of an abrasion, a cut, a bone fracture, and an open wound.

11. The method according to claim 1, wherein the peripherally-restricted kappa opioid receptor agonist is administered by a route of injection selected from the group consisting of subcutaneous injection, intravenous injection, intraperitoneal injection, intra-articular injection, intramuscular injection and intra-ocular injection.

12. The method according to claim 1, wherein the peripherally restricted kappa opioid receptor agonist comprises a peptide.

13. The method according to claim 2, wherein the peptide comprises at least four D-amino acids.

14. The method according to claim 1, wherein the peripherally restricted kappa opioid receptor agonist comprises a synthetic peptide amide having the formula: 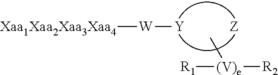
or a stereoisomer, mixture of stereoisomers, prodrug, pharmaceutically acceptable salt, hydrate, solvate, acid salt hydrate, N-oxide or isomorphic crystalline form thereof;
wherein
Xaa₁ is selected from the group consisting of (A)(A′)D-Phe, (A)(A′)(α-Me)D-Phe, D-Tyr, D-Tic, D-tert-leucine, D-neopentylglycine, D-phenylglycine, D-homophenylalanine, and β-(E)D-Ala, wherein each (A) and each (A′) are phenyl ring substituents independently selected from the group consisting of —H, —F, —Cl, —NO₂, —CH₃, —CF₃, —CN, and —CONH₂, and wherein each (E) is independently selected from the group consisting of cyclobutyl, cyclopentyl, cyclohexyl, pyridyl, thienyl and thiazolyl;
Xaa₂ is selected from the group consisting of (A)(A′)D-Phe, 3,4-dichloro-D-Phe, (A)(A′)(α-Me)D-Phe, D-1Nal, D-2Nal, D-Tyr, (E)D-Ala and D-Trp;
Xaa₃ is selected from the group consisting of D-Nle, D-Phe, (E)D-Ala, D-Leu, (α-Me)D-Leu, D-Hle, D-Val, and D-Met;
Xaa₄ is selected from the group consisting of (B)₂D-Arg, (B)₂D-Nar, (B)₂D-Har, ζ-(B)D-Hlys, D-Dap, ε-(B)D-Lys, ε-(B)₂-D-Lys, D-Amf, amidino-D-Amf, γ-(B)₂D-Dbu, δ-(B)₂α-(B′)D-Orn, D-2-amino-3(4-piperidyl)propionic acid, D-2-amino-3(2-aminopyrrolidyl)propionic acid, D-α-amino-β-amidinopropionic acid, α-amino-4-piperidineacetic acid, cis-α,4-diaminocyclohexane acetic acid, trans-α,4-diaminocyclohexaneacetic acid, cis-α-amino-4-methylaminocyclo-hexane acetic acid, trans-α-amino-4-methylaminocyclohexane acetic acid, α-amino-1-amidino-4-piperidineacetic acid, cis-α-amino-4-guanidinocyclohexane acetic acid, and trans-α-amino-4-guanidinocyclohexane acetic acid, wherein each (B) is independently selected from the group consisting of H and C₁-C₄ alkyl, and (B′) is H or (α-Me);
W is selected from the group consisting of:
Null, provided that when W is null, Y is N;
—NH—(CH₂)_{b}— with b equal to zero, 1, 2, 3, 4, 5, or 6; and
—NH—(CH₂)_{c}—O— with c equal to 2, or 3, provided that Y is C;
the moiety 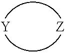
is an optionally substituted 4 to 8-membered heterocyclic ring moiety wherein all ring heteroatoms in said ring moiety are N; wherein Y and Z are each independently C or N; provided that when such ring moiety is a six, seven or eight-membered ring, Y and Z are separated by at least two ring atoms; and provided that when such ring moiety has a single ring heteroatom which is N, then such ring moiety is non-aromatic;
V is C₁-C₆ alkyl, and e is zero or 1, wherein when e is zero, then V is null and R₁ and R₂ are directly bonded to the same or different ring atoms;
wherein (i) R₁ is selected from the group consisting of —H, —OH, halo, —CF₃, —NH₂, —COOH, C₁-C₆ alkyl, C₁-C₆ alkoxy, amidino, C₁-C₆ alkyl-substituted amidino, aryl, optionally substituted heterocyclyl, Pro-amide, Pro, Gly, Ala, Val, Leu, Ile, Lys, Arg, Orn, Ser, Thr, —CN, —CONH₂, —COR′, —SO₂R′, —CONR′R″, —NHCOR′, OR′ and SO₂NR′R″; wherein said optionally substituted heterocyclyl is optionally singly or doubly substituted with substituents independently selected from the group consisting of C₁-C₆ alkyl, C₁-C₆ alkoxy, oxo, —OH, —Cl, —F, —NH₂, —NO₂, —CN, —COOH, and amidino; wherein R′ and R″ are each independently —H, C₁-C₈ alkyl, aryl, or heterocyclyl or R′ and R″ are combined to form a 4- to 8-membered ring, which ring is optionally singly or doubly substituted with substituents independently selected from the group consisting of C₁-C₆ alkyl, —C₁-C₆ alkoxy, —OH, —Cl, —F, —NH₂, —NO₂, —CN, —COOH and amidino; and R₂ is selected from the group consisting of —H, amidino, singly or doubly C₁-C₆ alkyl-substituted amidino, —CN, —CONH₂, —CONR′R″, —NHCOR′, —SO₂NR′R″ and —COOH; or
(ii) R₁ and R₂ taken together can form an optionally substituted 4- to 9-membered heterocyclic monocyclic or bicyclic ring moiety which is bonded to a single ring atom of the Y and Z-containing ring moiety; or
(iii) R₁ and R₂ taken together with a single ring atom of the Y and Z-containing ring moiety can form an optionally substituted 4- to 8-membered heterocyclic ring moiety to form a spiro structure; or
(iv) R₁ and R₂ taken together with two or more adjacent ring atoms of the Y and Z-containing ring moiety can form an optionally substituted 4- to 9-membered heterocyclic monocyclic or bicyclic ring moiety fused to the Y and Z-containing ring moiety;
wherein each of said optionally substituted 4-, 5-, 6-, 7-, 8- and 9-membered heterocyclic ring moieties comprising R₁ and R₂ is optionally singly or doubly substituted with substituents independently selected from the group consisting of C₁-C₆ alkyl, C₁-C₆ alkoxy, optionally substituted phenyl, oxo, —OH, —Cl, —F, —NH₂, —NO₂, —CN, —COOH, and amidino;
provided that when the Y and Z-containing ring moiety is a six or seven membered ring having a single ring heteroatom and e is zero, then R₁ is not —OH, and R₁ and R₂ are not both —H; and
provided further that when the Y and Z-containing ring moiety is a six membered ring having two ring heteroatoms, both Y and Z are N and W is null, then -(V)ₑR₁R₂ is attached to a ring atom other than Z; and if e is zero, then R₁ and R₂ are not both —H.

15. The method of claim 14, wherein the moiety: 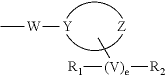
is selected from the group consisting of:

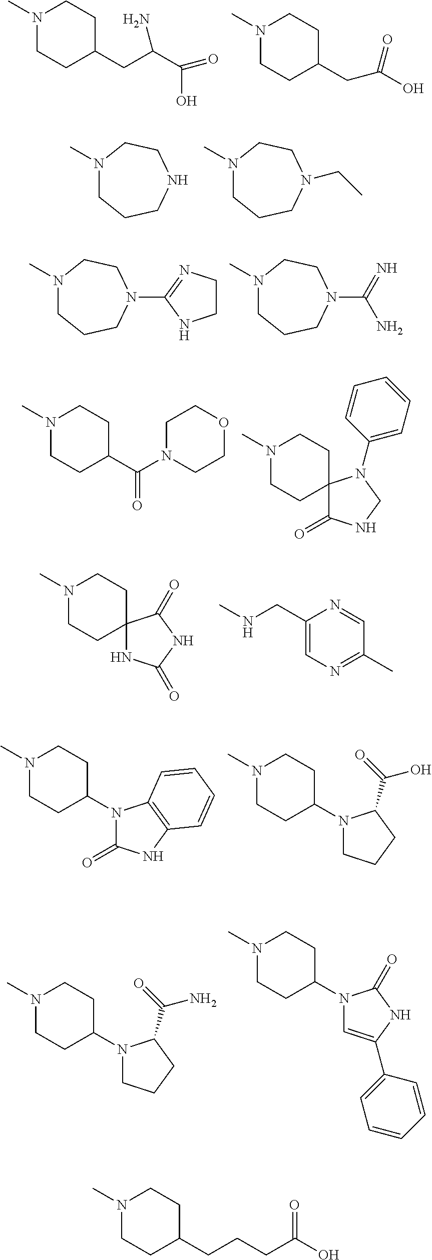

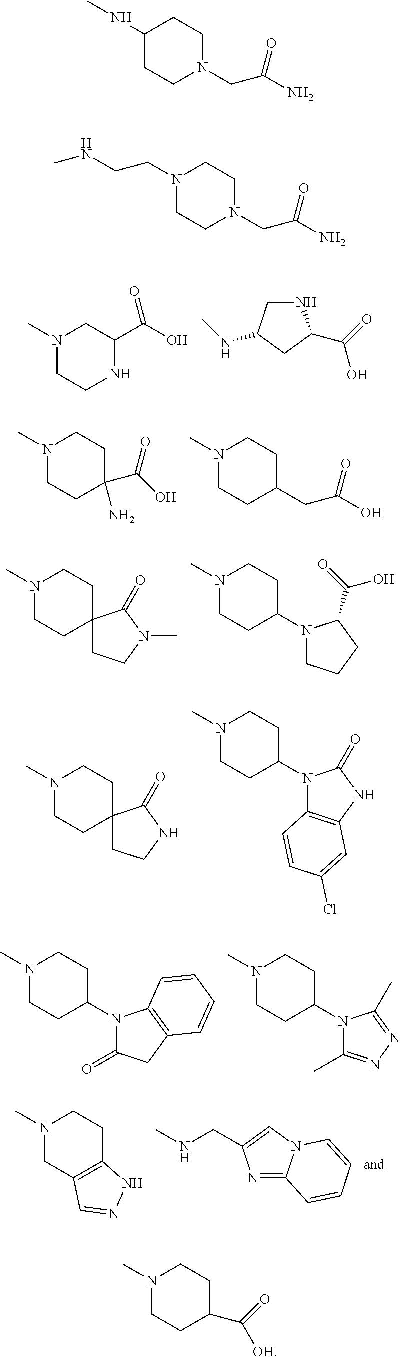

16. The method of claim 14, wherein the synthetic peptide amide has the structure:

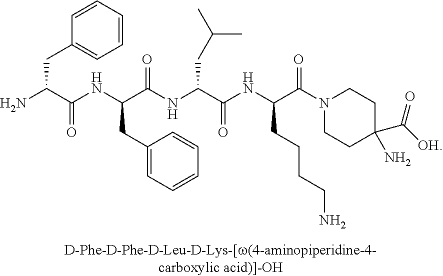 [SMILES: CC(C)C[C@@H](NC(=O)[C@@H](Cc1ccccc1)NC(=O)[C@H](N)Cc1ccccc1)C(=O)N[C@H](CCCCN)C(=O)N1CCC(N)(C(=O)O)CC1]

17. The method of claim 16, wherein the mammalian subject is a human.

18. The method according to claim 1, wherein the peripherally-restricted kappa opioid receptor agonist is a non-narcotic analgesic.

19. The method according to claim 1, wherein the peripherally-restricted kappa opioid receptor agonist is asimadoline (N-[(1S)-2-[(3S)-3-hydroxypyrrolidin-1-yl]-1-phenylethyl]-N-methyl-2,2-diphenylacetamide).

20. The method according to claim 1, wherein the peripherally-restricted kappa opioid receptor agonist is nalfurafine ((2E)-N-[(5α,6β)-17-(cyclopropylmethyl)-3,14-dihydroxy-4,5-epoxymorphinan-6-yl]-3-(3-furyl)-N-methylacrylamide).
